(19) United States
(12) Patent Application Publication
(10) Pub. No.: US 2008/0089942 A1
(43) Pub. Date: Apr. 17, 2008

(54) USE OF ADSORBENT CARBON MICROSPHERES TO TREAT INTESTINAL BACTERIAL INFECTIONS

(76) Inventors: Xavier Frapaise, Rancho Santa Fe, CA (US); Keith Anderson, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/840,035

(22) Filed: Aug. 16, 2007

Related U.S. Application Data
(60) Provisional application No. 60/838,931, filed on Aug. 17, 2006.

(51) Int. Cl. A61K 9/14; A61K 47/00

(52) U.S. Cl. 424/489; 514/789

(57) ABSTRACT

Disclosed herein is the use of adsorbent carbon microspheres for the treatment of intestinal bacterial infections. The infections include infection with Escherichia coli, Shigella dysenteriae, Vibrio cholerae, and Clostridium difficile. The adsorbent carbon microspheres serve to adsorb or bind toxins produced by the bacteria.

### RELATED APPLICATION

This application claims priority under 35 U.S.C. 119(e) to U.S. Provisional Application No. 60/838,931, filed Aug. 17, 2006, the entire contents of which are incorporated herein by reference.

### BACKGROUND

1. Field of the Invention

One aspect of the present invention relates to the treatment of intestinal bacterial infections and symptoms associated with such infections using adsorbent carbon micro spheres.

2. Description of the Related Art

Many bacterial infections target the intestines of a patient and cause disease through their ability to produce toxins. Bacterially produced toxins can be considered some of the most potent poisons known to man. Common toxin-producing bacteria that can infect human intestines include Escherichia coli, Shigella dysenteriae, Vibrio cholerae, and Clostridium difficile. Due to the continuing problems caused by these and other toxin-producing bacteria in both hospital and community settings, there is need for improved methods of treatment.

Escherichia coli can generally cause several intestinal and extra-intestinal infections. Certain strains of E. coli, such as Escherichia coli O157:H7, Escherichia coli O121 and Escherichia coli O104:H21, are toxigenic (some produce a toxin very similar to that seen in dysentery). They can cause food poisoning usually associated with eating cheese and contaminated meat. O157:H7 is further notorious for causing serious, even life threatening complications like HUS (Hemolytic Uremic Syndrome). Severity of the illness varies considerably but it can be fatal, particularly to young children, the elderly, or the immunocompromised.

Shigella dysenteriae is a Gram-negative, non-motile, non-spore forming rod-shaped bacteria closely related to Escherichia coli and Salmonella. S. dysenteriae is the causative agent of human shigellosis. Infection is typically via ingestion (e.g., fecal-oral contamination). S. dysenteriae causes dysentery that results in the destruction of the epithelial cells of the intestinal mucosa in the cecum and rectum. Some strains produce enterotoxin and Shiga toxin. Shiga toxin is associated with causing hemolytic uremic syndrome.

S. dysenteriae invade the host through epithelial cells of the small intestine. The most common symptoms are diarrhea, fever, nausea, vomiting, stomach cramps, and straining to have a bowel movement. The stool may contain blood, mucus, or pus. In rare cases, young children may have seizures. Symptoms can take as long as a week to show up, but most often begin two to four days after ingestion. Symptoms usually last for several days, but can last for weeks. Shigella is implicated as one of the pathogenic causes of reactive arthritis worldwide.

Vibrio cholerae is a Gram-negative bacteria that is the causative agent of cholera. It can be carried by numerous sea living animals, such as crabs or prawns, and has been known to cause fatal infections in humans during exposure. V. cholerae produces the toxin known as Cholera Toxin.

Clostridium difficile infection is typically acquired during antimicrobial use that causes disruption of the normal colonic flora. Upon ingestion, C. difficile spores vegetate, multiply, and secrete toxins in the colon that cause diarrhea and pseudomembranous colitis. The diarrhea is typically watery and occasionally bloody. C. difficile infections typically occur in hospitals and nursing homes. The principle toxins excreted by C. difficile include toxin A, an enterotoxin, and toxin B, a cytotoxin.

### SUMMARY

One embodiment disclosed herein includes a method of treating one or more symptoms of an intestinal bacterial infection, comprising administering to a subject adsorbent carbon microspheres having a particle size of about 0.01 to about 2 mm.

Another embodiment disclosed herein includes a method of reducing the level of a bacterial toxin in a colon, the method comprising introducing into the colon adsorbent carbon microspheres having a particle size of about 0.01 to about 2 mm.

Another embodiment disclosed herein includes a method of treating an intestinal bacterial infection, comprising co-administering to a subject an antibiotic and adsorbent carbon microspheres having a particle size of about 0.01 to about 2 mm.

### BRIEF DESCRIPTION OF THE DRAWINGS

FIG. 1 is a graph depicting bacterial toxin adsorption by adsorbent carbon microspheres as a function of time for low-levels of toxin production.

FIG. 2 is a graph depicting bacterial toxin adsorption by adsorbent carbon microspheres as a function of time for mid-levels of toxin production.

FIG. 3 is a graph depicting bacterial toxin adsorption by adsorbent carbon microspheres as a function of time for high-levels of toxin production.

### DETAILED DESCRIPTION

Definitions

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional such as a Medical Doctor (i.e. Doctor of Allopathic medicine or Doctor of Osteopathic medicine). or a Doctor of Veterinary Medicine to attempt to cure, or at least ameliorate the effects of, a particular disease or disorder or to prevent the disease or disorder from occurring in the first place.

As used herein, a “dosage” refers to an amount of therapeutic agent administered to a patient.

As used herein, a “daily dosage” refers to the total amount of therapeutic agent administered to a patient in a day.

As used herein, the term meq means milliequivalents(s).

As used herein, the term “therapeutic agent” means a substance that is effective in the treatment of a disease or condition. For example, the therapeutic agent(s) can be antibiotics.

Treatment of Bacterial Infections

In some embodiments, intestinal bacterial infections or symptoms associated with such infections are treated by administering adsorbent carbon microspheres to a patient. In some embodiments, the bacteria are toxin-producing bacteria. Non-limiting examples of such bacteria include Escherichia coli, Shigella dysenteriae, Vibrio cholerae, and Clostridium difficile. As used herein, an “adsorbent carbon microsphere” is a particle having a spherical or spheroid-like shape whose composition is mostly carbon and which has adsorbent properties. In some embodiments, the carbon microspheres have diameters from about 0.01 to about 2 mm. In some embodiments, the diameters are from about 0.02 to about 1 mm. In still other embodiments, the diameters are from about 0.05 to about 0.8 mm. In a typical embodiment, the carbon microspheres can have diameters from about 0.1 to about 0.5 mm. For example, the carbon microspheres can have diameters from about 0.2 to about 0.4 mm.

In some embodiments, the adsorbent carbon microspheres have a specific surface area of about 700 m²/g or more, such as determined by a BET (Brunauer-Emmett-Teller theory model) method (Brunauer et al. “Adsorption of Gases in Multimolecular Layers”, J. Am. Chem. Soc., 1938, 60(2), 309-319, which is incorporated herein by reference in its entirety). In some embodiments, the specific surface area is from about 700 m²/g to about 2500 m²/g. In a typical embodiment, the specific surface area is from about 1400 m²/g to about 1900 m²/g. For example, the specific surface area may be from about 1500 m²/g to about 1800 m²/g. In some embodiments, the volume of pores in the carbon microspheres having a pore diameter of about 20 to about 15,000 nm are from about 0.04 mL/g to about 0.10 mL/g.

In some embodiments, the total amount of acidic groups on the carbon are from about 0.30 to about 1.20 meq/g. In a typical embodiment, the total amount of acidic groups on the carbon is from about 0.30 to about 0.80 meq/g. For example, the total amount of acidic groups on the carbon may be from about 0.40 to about 0.70 meq/g. In some embodiments, the total amount of basic groups on the carbon are from about 0.20 to about 1.00 meq/g. In a typical embodiment, the total amount of acidic groups on the carbon can be from about 0.30 to about 0.80 meq/g. For example, the total amount of acidic groups on the carbon may be from about 0.35 to about 0.65 meq/g.

Suitable forms of adsorbent carbon microspheres are also described in U.S. Pat. Nos. 4,681,764 and 6,830,753 and U.S. Application Publication Nos. 2005/0112114; 2005/0079167; and 2005/0152890; all of which are incorporated herein by reference in their entirety.

In one embodiment, the adsorbent carbon microspheres are AST-120, available under the trade name KREMEZIN® from Kureha Corp. (Japan). AST-120 is a spherical activated carbon produced from pitch, such as by the process disclosed in U.S. Pat. No. 4,681,764. AST-120 has a particle size of about 0.2 to about 0.4 mm and is a homogeneous spherical particle (not a spherical particle produced by granulating a carbon powder).

While not being bound by any particular theory, it is believed that adsorbent carbon microspheres as described above act to treat symptoms of intestinal bacterial infections by adsorption of bacterial toxins at the site of infection. Non-limiting examples of such bacterial toxins include enterotoxins (E. coli), type 1 and type 2 Shiga toxins (S. dysenteriae), Cholera Toxin (V. cholerae) and Toxin A (C. difficile). In some embodiments, administration of adsorbent carbon microspheres to a patient suffering from an intestinal bacterial infection results in a decrease in one or more symptom caused by the infection, including but not limited to diarrhea, fever, nausea, vomiting, stomach cramps, straining to have a bowel movement, and blood or mucus in stool. In some embodiments, the adsorbent carbon microspheres reduce the incidence of death in patients suffering from infection. In some embodiments, the adsorbent carbon microspheres are effective without causing certain side effects such as constipation or adsorption of beneficial intestinal enzymes (e.g., α-amylase).

In some embodiments, adsorbent carbon microspheres such as AST-120 are administered in combination with one or more additional therapeutic agent(s) to treat bacterial infections. In some embodiments, the additional therapeutic agent is an antibacterial agent including but not limited to quinolones, tetracyclines, glycopeptides, aminoglycosides, β-lactams, rifamycins, macrolides/ketolides, oxazolidinones, coumermycins, and chloramphenicol.

Non-limiting examples of β-lactams include imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephaacetrile, cephalexin, cephaloglycin, cephaloridine, cephalothin, cephapirin, cephradine, cefmetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, and LY206763.

Non-limiting examples of macrolides include azithromycin, clarithromycin, erythromycin, oleandomycin, rokitamycin, rosaramicin, roxithromycin, and troleandomycin.

Non-limiting examples of ketolides include telithromycin and cethrimycin.

Non-limiting examples of quinolones include amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin, gemifloxacin, garenoxacin, olamufloxacin, clinofloxacin, trovafloxacin, balofloxacin, prulifloxacin, moxifloxacin, gemifloxacin, rufloxacin, sitafloxacin (Sato, K, et al., 1992, Antimicrob Agents Chemother. 37:1491-98, which is incorporated herein by reference in its entirety), marbofloxacin, orbifloxacin, sarafloxacin, danofloxacin, difloxacin, enrofloxacin, TG-873870, DX-619, DW-276, ABT-492, DV-7751a (Tanaka, M, et al., 1992, Antimicrob. Agents Chemother. 37:2212-18), and F-1061 (Kurosaka et al., Interscience Conference on Antimicrobial Agents and Chemotherapy, 2003, 43rd: Chicago, which is incorporated herein by reference in its entirety).

Non-limiting examples of tetracyclines, glycylcyclines, and oxazolidinones include chlortetracycline, demeclocycline, doxycycline, lymecycline, methacycline, minocycline, oxytetracycline, tetracycline, tigecycline, linezolide, and eperozolid.

Non-limiting examples of aminoglycosides include amikacin, arbekacin, butirosin, dibekacin, fortimicins, gentamicin, kanamycin, meomycin, netilmicin, ribostamycin, sisomicin, spectinomycin, streptomycin, and tobramycin.

Non-limiting examples of lincosamides include clindamycin and lincomycin.

The oral dosage of the adsorbent carbon microspheres, either administered alone or in combination with another therapeutic agent, may be from about 1 to 20 grams per day. In some embodiments, the daily dosage of the adsorbent carbon microspheres may be divided into multiple administrations (e.g. into two to four portions daily). In some embodiments each portion can be from about 1 g to about 5 g (e.g., about 2 g to about 3 g). In a typical embodiment, a patient takes from about 2 g to about 3 g of adsorbent carbon microspheres (e.g., AST-120) three times daily.

In some embodiments, the adsorbent carbon microspheres can be prescribed or administered at a specific dosage per day. In other embodiments, the patient can be instructed to take the composition when he or she experiences one or more symptoms related to a condition being treated.

As used herein, by administration in “combination,” it is meant that the adsorbent carbon microspheres are in the patient at the same time as one or more therapeutic agents may be found in the patient's bloodstream or stomach, regardless of when or how the adsorbent carbon microspheres and therapeutic agents are actually administered. In one embodiment, the adsorbent carbon microspheres and the therapeutic agent(s) are administered simultaneously. In one such embodiment, administration in combination is accomplished by combining the adsorbent carbon microspheres and the therapeutic agent(s) in a single dosage form. In another embodiment, the adsorbent carbon microspheres and the therapeutic agent(s) are administered sequentially. In one embodiment the adsorbent carbon microspheres and the therapeutic agent(s) are administered through the same route, such as orally. In another embodiment, the adsorbent carbon microspheres and the therapeutic agent(s) are administered through different routes, such as one being administered orally and another being administered i.v.

Production of Adsorbent Carbon Microspheres

Adsorbent carbon microspheres suitable for use as described herein may be produced by any suitable method, including but not limited to the following:

First, a bicyclic or tricyclic aromatic compound or a mixture thereof having a boiling point of 200° C. or more is added as an additive to a pitch such as a petroleum pitch or a coal pitch. The whole is heated and mixed, and then shaped to obtain a shaped pitch. Thereafter, the shaped pitch is dispersed and granulated in hot water at 70 to 180° C., with stirring, to obtain a microspherical shaped pitch. The aromatic additive is extracted and removed from the shaped pitch by a solvent having a low solubility to the pitch but a high solubility to the additive. The resulting porous pitch is oxidized by an oxidizing agent to obtain a porous pitch subject to heat infusibility. The resulting infusible porous pitch is treated at 800 to 1000° C. in a gas flow such as steam or carbon dioxide gas reactive with carbon to obtain a porous carbonaceous substance.

The resulting porous carbonaceous substance is then oxidized in a temperature range of 300 to 800° C., preferably 320 to 600° C., in an atmosphere containing 0.1 to 50% by volume, preferably 1 to 30% by volume, particularly preferably 3 to 20% by volume, of oxygen. The substance is thereafter reduced in a temperature range of 800 to 1200° C., preferably 800 to 1000° C., in an atmosphere of a non-oxidizable gas to obtain the final product.

In one alternative, the adsorbent carbon microspheres may be produced from a resin instead of a pitch. More details of suitable production processes and suitable products may be found in U.S. Pat. Nos. 4,681,764; 6,830,753; and U.S. Application Publication No. 2005/0112114, filed May 26, 2005, all of which are incorporated herein by reference in their entirety. Suitable adsorbent carbon microspheres are commercially available from Kureha Corp., and are sold in Japan under the trade name KREMEZIN® (also known as AST-120).

Pharmaceutical Compositions

For use as described herein, adsorbent carbon microspheres may be administered to the gut of a subject by any suitable means. In one embodiment, the adsorbent carbon microspheres are administered orally. Formulations for oral administration may include, but are not limited to, the free flowing microspheres, granules, tablets, sugar-coated tablets, capsules, suspensions, sticks, divided packages, or emulsions. In the case of capsules, gelatin capsules, or if necessary, enteric capsules may be used. In the case of tablets, the formulations may advantageously be adapted to break into the original fine particles inside the body. In the case of free flowing microspheres, the formulations may be in a sachet that is opened immediately prior to ingestion. The adsorbent may be used as a mixture with an electrolyte-controlling agent, such as an aluminum gel or KAYEXALATE® (Winthrop Lab, U.S.A.) or other agents. The microspheres may be ingested with the aid of a liquid or soft food (e.g., apple sauce).

Certain preparations for oral use can be obtained by mixing one or more excipients with adsorbent carbon microspheres as described herein and processing the mixture after adding suitable auxiliaries, if desired, to obtain tablets or dragee cores. The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration.

Excipients can include, by way of illustration and not limitation, diluents, disintegrants, binding agents, adhesives, wetting agents, polymers, lubricants, glidants; substances added to mask or counteract a disagreeable texture, taste or odor; flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

A variety of techniques for formulation and administration can be found in Remington: The Science and Practice of Pharmacy (20^{th} ed, Lippincott Williams & Wilkens Publishers (2003)), which is incorporated herein by reference in its entirety.

The dosage form used may be any amount of adsorbent carbon microspheres suitable to achieve the desired therapeutic result. In some embodiments, each unit dose is from about 1 g to about 5 g (e.g., about 2 g to about 3 g). In some embodiments, dosages of the adsorbent are individually packaged so as to preserve the adsorptivity of the material. For example, divided packaging may be used such as described in more detail in U.S. Pat. No. 5,686,081, which is incorporated herein by reference in its entirety. The divided packaging may contain unit doses of the adsorbent carbon microspheres in their free flowing form.

### EXAMPLES

### Example 1

A human patient suffering from a C. difficile infection is administered adsorbent carbon microspheres having particle sizes between about 0.05 and about 2 mm. The adsorbent carbon microspheres are administered orally as 2 g of free flowing microspheres three times daily. The symptoms of the C. difficile infection are reduced after repeated administration of the carbon microspheres.

### Example 2

A human patient is suffering from diarrhea. A stool analysis indicates the presences of toxins A and/or B in the stool. Adsorbent carbon microspheres having particle sizes between about 0.05 and about 2 mm are administered orally as 2 g of free flowing microspheres three times daily. A subsequent stool analysis indicates a reduction in the levels of toxins A and/or B.

### Example 3

A known amount of toxin A dissolved in an appropriate buffer is mixed with adsorbent carbon microspheres having particle sizes between about 0.05 and about 2 mm for a period of time. The remaining amount of toxin A in solution is determined using an appropriate assay. The results indicate that the level of toxin A in solution has decreased.

### Example 4

A known amount of toxin B dissolved in an appropriate buffer is mixed with adsorbent carbon microspheres having particle sizes between about 0.05 and about 2 mm for a period of time. The remaining amount of toxin B in solution is determined using an appropriate assay. The results indicate that the level of toxin B in solution has decreased.

### Example 5

The ability of AST-120 to bind or adsorb four bacterial toxins was tested. The toxins included Escherichia coli (E. coli) heat stable enterotoxin (STa), Shigella dysenteriae (S. dysenteriae) type 1 Shiga toxin (Stx), Vibrio cholerae (V. cholerae) Cholera Toxin (CT), and Clostridium difficile (C. difficile) Toxin A.

Strains

E. coli, S. dysenteriae, V. cholerae, and C. difficile were obtained from American Type Culture Collection (Manassas, Va.). The strains were chosen based on their ability to produce the desired toxin targeted for testing. All culturing was conducted in media obtained from BD (Sparks, Md.) and confirmed through testing to be suitable for growth and subsequent toxin production for the various pathogens.

Test/Control Articles

The test article, AST-120, was obtained from QCL, Inc (Wilmington, N.C.). The control article, microcrystalline cellulose spheres, was obtained from Schwarz Pharma Mfg., Inc (Seymour, Ind.). All testing was conducted using Phosphate-Buffered Saline (PBS) obtained from Invitrogen (Carlsbad, Calif.).

Toxin Detection Kits

The test kits utilized for confirming toxin production in culture filtrates and supernatants were obtained from Remel, Inc. (Lenexa, Kans.). The EIA kit, a competitive enzyme immunoassay (EIA), was used to detect STa. The VTEC-RPLA kit, a reverse passive latex agglutination test, was used to detect Stx. The VET-RPLA kit, a reverse passive latex agglutination test, was used to detect CT. The PET-RPLA kit, a reverse passive latex agglutination test, was used to detect Toxin A.

Toxin-Binding Testing

Cultures of E. coli, S. dysenteriae and V. cholerae in Tryptic Soy Broth (TSB) were incubated overnight with shaking at 37° C. in an aerobic atmosphere. Cultures of C. difficile in Reinforced Clostridial Medium (RCM) were incubated for 48 hours at 37° C. in an anaerobic atmosphere. Based on the sensitivity of the test kits in regards to the lower limits of toxin detection, the minimal concentration of organism needed to produce toxin was determined. Aliquots were taken at varying time points and read at an absorbance reading of 650 (O.D.₆₅₀). These numbers represented what was considered for testing purposes as low-level toxin production. Based on the growth characteristics of the various organisms, the O.D. readings were used as a means to extrapolate numbers to represent mid-level and high-level toxin production. The relationship between O.D. reading and concentration from the extrapolation is presented in Table 1.

TABLE 1
Corresponding O.D. readings and toxin levels expressed in ng/ml.

| | Low-level | | Mid-level | | High-level | |
|---|---|---|---|---|---|---|
| Toxin | (O.D. = Conc.) | | (O.D. = Conc.) | | (O.D. = Conc.) | |
| STa | 5.0 = 10 | ng/ml | 6.5 = 13 | ng/ml | 8.0 = 16 | ng/ml |
| Stx | 0.45 = 1-2 | ng/ml | 1.5 = 3-6 | ng/ml | 5.0 = 11-22 | ng/ml |
| CT | 0.9 = 1-2 | ng/ml | 3.0 = 3-6 | ng/ml | 5.0 = 6-11 | ng/ml |
| Toxin A | 2.0 = 2 | ng/ml | 3.0 = 3 | ng/ml | 4.0 = 4 | ng/ml |

Once cultures reached the desired density, they were centrifuged at 900 g for 20-30 minutes at 4° C. and the supernatants were resuspended in PBS to a 25 mL volume. Aliquots of AST-120 were allowed to dry for 4 hours at 105° C. After drying, a concentration of 0.25 g AST-120 was added to each 25 mL PBS/toxin combinations in 500 mL flasks. The flasks were incubated with shaking (78 rpm) at 37° C. Aliquots (0.5 mL) were removed hourly over a 6 hour time period and filtered using 0.65 um low protein-binding filters. The filtrates were then used to inoculate the test wells contained within the toxin detection test kits. Duplicate testing was always conducted using the control article, where no binding ever occurred.

Results

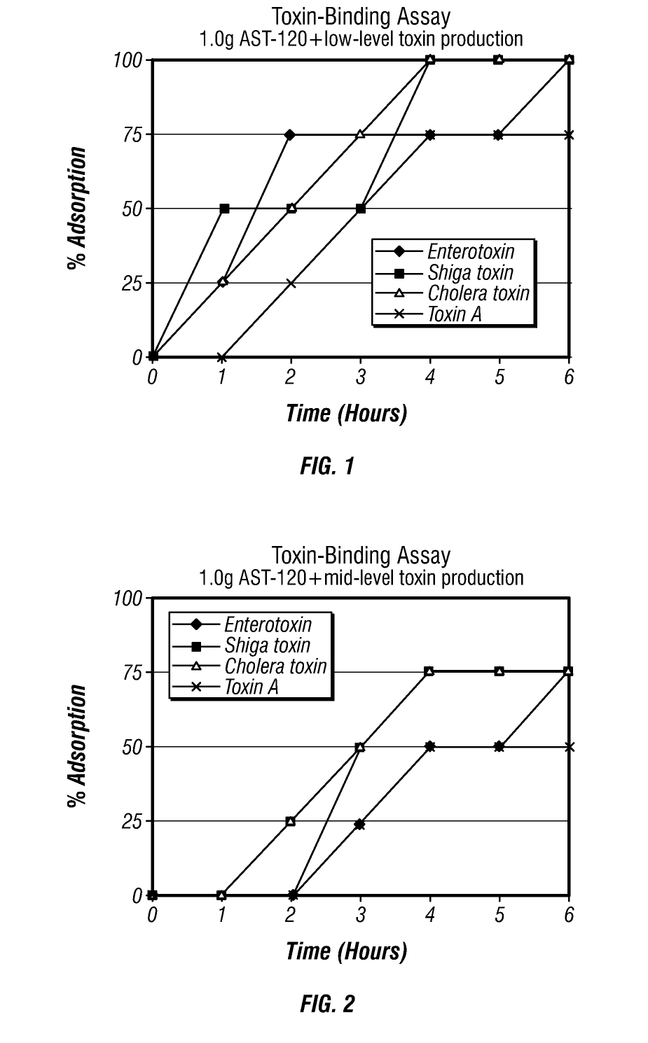

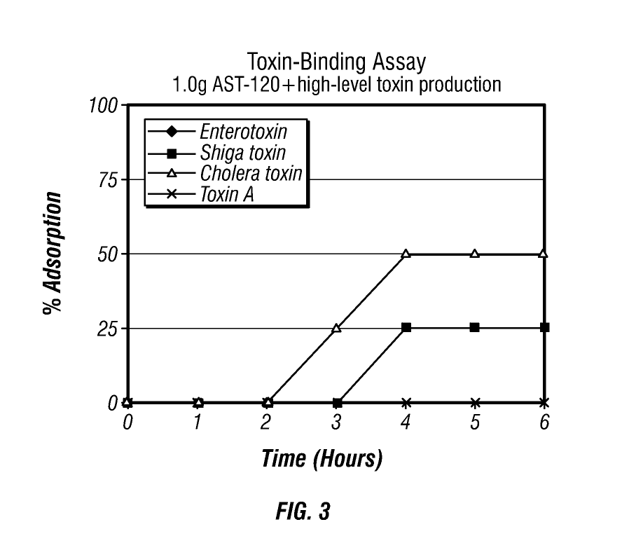

Various studies were conducted to optimize the testing parameters used to evaluate the toxin binding or adsorption efficacy of AST-120 in vitro. After optimization, the adsorption efficacy was evaluated as a function of time for low, mid, and high levels of toxin production. FIG. 1 is a graph depicting the measured adsorption as a function of time for low-levels of toxin production. FIG. 2 is a graph depicting the measured adsorption as a function of time for mid-levels of toxin production. FIG. 3 is a graph depicting the measured adsorption as a function of time for high-levels of toxin production. The results indicated that AST-120 adsorption or binding ability of toxins STa, Stx, CT and Toxin A is both time and toxin concentration dependent, demonstrating that AST-120 is efficacious in its ability to bind the toxins.

Although the invention has been described with reference to embodiments and examples, it should be understood that numerous and various modifications can be made without departing from the spirit of the invention. Accordingly, the invention is limited only by the following claims.

What is claimed is:

1. A method of treating one or more symptoms of an intestinal bacterial infection, comprising administering to a subject adsorbent carbon microspheres having a particle size of about 0.01 to about 2 mm.

2. The method of claim 1, further comprising identifying the subject as suffering from an intestinal bacterial infection.

3. The method of claim 1, wherein the bacteria is selected from the group consisting of Escherichia coli, Shigella dysenteriae, Vibrio cholerae, and Clostridium difficile.

4. The method of claim 1, wherein the bacterial infection produces a bacterial toxin.

5. The method of claim 4, wherein the toxin is selected from one or more of heat stable enterotoxin, type 1 Shiga toxin, type 2 Shiga toxin, Cholera Toxin, and Toxin A.

6. The method of claim 4, wherein the toxin is selected from one or more of heat labile enterotoxin and Toxin B.

7. The method of claim 1, wherein the symptoms are selected from one or more of diarrhea, fever, nausea, vomiting, stomach cramps, straining to have a bowel movement, and blood or mucus in stool.

8. The method of claim 1, wherein the adsorbent carbon microspheres are administered orally in a free flowing form.

9. The method of claim 1, wherein from about 1 gram to about 20 grams of the adsorbent carbon microspheres is administered daily.

10. The method of claim 1, wherein the adsorbent carbon microspheres are administered three times daily.

11. The method of claim 1, wherein each dose of adsorbent carbon microspheres administered is from about 2 grams to about 3 grams.

12. The method of claim 1, wherein the adsorbent carbon microspheres have a particle size of about 0.02 to about 1 mm.

13. The method of claim 1, wherein the adsorbent carbon microspheres have a particle size of about 0.05 to about 0.8 mm.

14. The method of claim 1, wherein the adsorbent carbon microspheres have a specific surface area of about 700 m²/g or more as determined by a BET (Brunauer-Emmett-Teller) method.

15. The method of claim 1, wherein the adsorbent carbon microspheres have a specific surface area of about 700 m²/g to about 2500 m²/g as determined by a BET (Brunauer-Emmett-Teller) method.

16. The method of claim 1, wherein the volume of pores in the adsorbent carbon microspheres having a pore diameter of about 20 to about 15,000 nm is about 0.04 mL/g to about 0.10 mL/g.

17. The method of claim 1, wherein the total amount of acidic groups in the adsorbent carbon microspheres is from about 0.30 to about 1.20 meq/g.

18. The method of claim 1, wherein the total amount of basic groups in the adsorbent carbon microspheres is from about 0.20 to about 1.00 meq/g.

19. A method of reducing the level of a bacterial toxin in a colon, the method comprising introducing into the colon adsorbent carbon microspheres having a particle size of about 0.01 to about 2 mm.

20. The method of claim 19, wherein the colon is in a human and the method further comprises identifying the human as having the bacterial toxin in the colon.

21. The method of claim 19, wherein the toxin is selected from one or more of heat stable enterotoxin, type 1 Shiga toxin, type 2 Shiga toxin, Cholera Toxin, and Toxin A.

22. The method of claim 19, wherein the toxin is selected from one or more of heat labile enterotoxin and Toxin B.

23. The method of claim 19, wherein introducing the adsorbent carbon microspheres into the colon comprises administering the adsorbent carbon microspheres to a human comprising the colon.

24. The method of claim 23, wherein the adsorbent carbon microspheres are administered orally in a free flowing form.

25. A method of treating an intestinal bacterial infection, comprising co-administering to a subject an antibiotic and adsorbent carbon microspheres having a particle size of about 0.01 to about 2 mm.

26. The method of claim 25, wherein the antibiotic is selected from the group consisting of quinolones, tetracyclines, glycopeptides, aminoglycosides, β-lactams, rifamycins, macrolides/ketolides, oxazolidinones, coumermycins, and chloramphenicol.
